(19) United States
(12) Patent Application Publication
(10) Pub. No.: US 2016/0135829 A1
(43) Pub. Date: May 19, 2016

(54) SYSTEMS AND METHODS FOR EN BLOC REMOVAL OF UNDESIRABLE MATERIAL FROM PASSAGEWAYS

(71) Applicant: AngioDynamics, Inc., Latham, NY (US)

(72) Inventors: Kevin Holochwost, Hudson, MA (US); Paul DiCarlo, Middleboro, MA (US)

(73) Assignee: AngioDynamics, Inc., Latham, NY (US)

(21) Appl. No.: 14/940,289

(22) Filed: Nov. 13, 2015

Related U.S. Application Data
(60) Provisional application No. 62/079,115, filed on Nov. 13, 2014.

(51) Int. Cl. A61B 17/22

(52) U.S. Cl. CPC A61B 17/22032; A61B 2017/22079; A61B 2017/00292

(57) ABSTRACT

The present disclosure relates to systems and methods for removing obstructions or occlusions from vascular and non-vascular passageways. More particularly, the present disclosure relates to systems and methods for en bloc removal of soft fresh thrombi and large emboli within the circulatory system and reinfusing fluid back into the patient to minimize fluid loss.

### CROSS REFERENCE TO RELATED APPLICATIONS

The present application relies on and claims the benefit of the filing date of U.S. Provisional Application No. 62/079,115, filed on Nov. 13, 2014.

### FIELD

The present disclosure relates to systems and methods for removing obstructions or occlusions from vascular and non-vascular passageways. More particularly, the present disclosure relates to systems and methods for en bloc removal of soft fresh thrombi and large emboli within the circulatory system and reinfusing fluid back into the patient to minimize fluid loss. Although specific reference is made to medium and large vascular passageways, it should be appreciated that the systems and methods disclosed herein may be scaled and adapted for use within any passageway within the body.

### BACKGROUND

Thromboembolic disorders such as myocardial infarction, stroke, pulmonary embolism, deep vein thrombosis, atrial fibrillation, infective endocarditis and arteriosclerosis are a major cause of morbidity and mortality in the United States. Such disorders are characterized by the partial or complete occlusion of a blood vessel by clots, fatty deposits or plaque. The oxygen and nutrient deprivation resulting from such blockages often causes the tissues downstream of the occluded vessel to become ischemic. Failure to restore proper circulation may to loss of limbs, angina pectoris. myocardial infarction, stroke and/or death. For example, obstructions within a systemic artery (i.e., arterial ischemia) may interfere with the delivery of oxygen-rich blood to organs and tissues, ultimately leading to tissue death or infarction. Obstructions within a systemic vein (i.e., venous congestion) may interfere with the suction of oxygen-poor blood and fluid from organs and tissues, resulting in swelling (edema) that can also lead to tissue infarction.

The most effective treatment for conditions resulting from blood clots or other undesirable materials within the circulatory system is to stabilize or eliminate the material before it has embolized. Alternatively, if obstruction to blood flow has already occurred but not yet caused permanent damage, the occlusive material may be eliminated using a variety of biological or mechanical means. Mechanical treatments generally eliminate the occlusive material by direct manipulation using, for example, aspiration, maceration and/or compression against the vessel wall. A potential drawback to known mechanical thrombectomy devices is the unwanted potential for vessel dissection or vessel rupture. Catheter-based mechanical treatments are generally divided into three categories: 1) fragmentation thrombectomy, 2) rheolytic thrombectomy and 3) aspiration, Fragmentation thrombectomy breaks down the obstruction blood clot) into smaller pieces, most of which migrate further downstream, decreasing the central obstruction but resulting in a “no-reflow” phenomenon. Rheolytic thrombectomy utilizes high velocity saline jets to create a Venturi effect that breaks down the obstruction and then draws the fragments into the catheter. Finally, aspiration techniques draw the obstruction into a catheter using suction. A distinct advantage of mechanical treatments is that the occlusive material is directly engaged independent of the specific content of the material and the occlusive material is removed or otherwise dissolved so that flow can be reestablished. A primary reason for this limited success for the majority of currently known mechanical thrombectomy devices is due to their inability to achieve en bloc (i.e., complete; as a whole) removal of the material without fragmentation. Preventing fragmentation is critical since any portion of the material that breaks free and escapes removal by the thrombectomy device may travel to other locations, such as the brain or lung, with catastrophic consequences. For example, fragmented material that travels to the lung may lead to a pulmonary embolism, permanent lung damage and/or sudden death. Fragmented material that travels to the brain may cause a debilitating or fatal stroke.

There is a continued need for systems and methods for quick and efficient removal of undesirable materials, including, but not limited to, soft fresh thrombi and large emboli from medium to large blood vessels and heart chambers with minimal fragmentation and without excessive blood loss.

### SUMMARY

The present disclosure relates generally to systems and method for removing obstructions or occlusions from within vascular passageways.

In one aspect, the present. disclosure provides a system for removing an undesirable material from within a vessel, the apparatus comprising: a cannula having a proximal end, a distal end and a lumen extending there between; wherein a distal portion of the cannula is moveable between a first configuration and a second configuration larger than the first configuration; and wherein the distal portion of the cannula includes an inner wall comprising a first material having a first coefficient of friction and a second material having, a second coefficient of friction different than the first material, wherein the first and second materials are arranged in a spiral pattern. The distal portion of the cannula may be configured to generate vortex flow therethrough. The undesired material includes one of a clot, a thrombus and an embolus, and wherein the vortex flow facilitates en bloc removal of the undesirable material through the cannula. The distal portion of the cannula may be configured to engage and capture and undesirable material when in the second configuration. The second configuration of the distal portion of the cannula may include a flared-open or funnel-like shape. The distal portion of the cannula may move from the first configuration to the second configuration when a fluid flow rate through the cannula lumen decreases below a threshold value. The distal portion of the cannula may move from the first configuration to the second configuration when a pressure within the cannula lumen decreases below a threshold value. The distal portion of the cannula may return to the first configuration when a fluid flow rate through the cannula lumen returns to a threshold value. The distal portion of the cannula may return to the first configuration when a pressure within the cannula lumen returns to a threshold value. The distal portion of the cannula may include a dielectric polymer that responds to an electrical signal to move the distal portion of the cannula between the first and second configurations. The cannula may be formed from a sufficiently pliable material to permit maneuvering of the cannula within the vessel.

In another aspect, the present disclosure provides a system for removing an undesirable material from within a vessel, the apparatus comprising: a cannula having a proximal end, a distal end and a lumen extending therebetween; wherein a distal portion of the cannula is moveable between a first configuration and a second configuration larger than the first configuration; and wherein the distal portion of the cannula includes an inner surface comprising at least one spiral groove.

In another aspect, the present disclosure provides a system for removing an undesirable material from within a vessel, the system comprising: a first cannula having a proximal end, a distal end and a lumen extending therebetween; wherein a distal portion of the cannula is moveable between a first configuration and a second configuration larger than the first configuration; and wherein the distal portion of the cannula includes an inner wall comprising a first material having a first coefficient of friction and a second material having a second coefficient of friction different than the first material, wherein the first and second materials are arranged in a spiral pattern; a second cannula in fluid communication with the first cannula and having its distal end situated in spaced relation to the distal end of the first cannula, such that fluid removed from the site of interest by the first cannula can be directed along the second cannula and reinfused through the distal end of the second cannula; and a pump, in fluid communication with the proximal end of the first cannula, to provide a sufficient suction force for generating a vortex flow through the expanded distal and to permit removing the undesirable material from the site of interest, while in fluid communication with the second cannula, to provide a sufficient continuous reinfusion force so as to reintroduce the fluid flow removed from the site of interest.

In yet another aspect, the present disclosure provides a method for removing an undesirable material from within a vessel, the method comprising: maneuvering a first cannula having a distal end through which an undesirable material can be captured to a site of interest within a vessel of a patient, such that the distal end of the first cannula is positioned adjacent the undesirable material; positioning a second cannula, in fluid communication with the first cannula, such that its distal end is situated in spaced relation to the distal end of the cannula; providing a suction force to generate a vortex flow through the distal end of the first cannula, so as to remove the undesirable material en bloc from the first cannula; and while providing the suction force to the first cannula, continuously imparting a reinfusion force through the distal end of the second cannula, so that any fluid remove along with the undesirable material by the first cannula can be reintroduced by the second cannula to a location in spaced relation form the distal end of the first cannula. The distal end of the first cannula may expand from a first diameter to a second diameter larger than the first diameter to remove large embolic emboli.

### BRIEF DESCRIPTION OF THE DRAWINGS

Non-limiting embodiments of the present disclosure will be described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure, In the figures:

FIG. 1 depicts a side view of the distal portion of a suction cannula configured to establish vortex flow, according to one embodiment of the present disclosure.

FIGS. 2A-2B depict the en bloc removal of undesirable material from a vessel using the suction cannula of FIG. 1, according to an embodiment of the present disclosure.

FIG. 3 depicts the en bloc removal of undesirable material from a vessel using suction cannula that includes helical spiraling which extends the length of the suction cannula lumen, according to an embodiment of the present disclosure.

FIGS. 4A-4C depict the en bloc, removal of undesirable material from a vessel using a suction cannula that includes a variable-diameter aperture, according to an embodiment of the present disclosure.

FIGS. 5A-5C depict an expanded view of the variable-diameter aperture moving from an unexpanded (FIGS. SA and 5B) to expanded (FIG. 5C) configuration, according to an embodiment of the present disclosure

FIG. 6 depicts a cross-sectional view of the suction cannula of FIG. 1 taken along the line 6-6, according to an embodiment of the present disclosure.

FIG. 7 depicts a cross-sectional view of the radially inward fluid flow through the distal portion of the suction cannula of FIG. 1, according to an embodiment of the present disclosure.

FIGS. 8A-8B depict cross-sectional views of the suction cannula of FIG. 1 taken along the lines 8A-8A and 8B-8B, respectively, according to an embodiment of the present disclosure.

FIG. 9 depicts a schematic view of the electromechanical feedback loop which regulates the variable-diameter aperture of FIGS. 5A-SC, according to an embodiment of the present disclosure.

FIG. 10 is a flowchart summarizing the steps involved in removing undesirable material from a vessel using a suction cannula with a variable-diameter aperture, according to an embodiment of the present disclosure.

FIGS. 11A-11B depict the system of the present disclosure deployed within a patient for en bloc removal of an occlusion from within a chamber of the heart, according to an embodiment of the present disclosure.

### DETAILED DESCRIPTION OF THE PREFERRED EMBODIMENTS

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one or ordinary skill in the art to which the disclosure belongs.

As used herein, the term “distal” refers to the end farthest away from a medical professional when introducing a device into a patient, while the term “proximal” refers to the end closest to the medical professional when introducing a device into a patient.

As used herein, the term “expanded” or “flared-open” refers to an increase in size, diameter or profile as compared to the size, diameter or profile in an “unexpanded” or “collapsed” configuration.

As used herein, the term “undesirable material” refers to any natural or unnatural or man-made) obstruction or occlusion within a vascular or non-vascular passageway. Such undesirable materials may include, but are not limited to, blood clots, soft fresh thrombi, vegetative matter and/or large emboli. Vascular passageways from which such undesirable material(s) may be removed include, for example, any vasculature of the human body large enough to allow the device to be placed within its lumen, those within the pulmonary circulation (e.g. pulmonary arteries), systemic venous circulation (e.g., vena cavae, pelvic veins, leg veins, neck and arm veins) or arterial circulation (e.g., aorta or its large and medium branches). The heart chambers may include, for example, the left heart (e.g., the left ventricular apex and left atrial appendage), right heart right atrium and right ventricle) or the valves. Non-vascular passageways may include, for example, bodily tubes (nasal tubes, Eustachian tubes, fallopian tubes die) canals (e.g., alveolar canals, spinal canal, ear canal etc.) and/or ducts (e.g., hepatic ducts, bile ducts, pancreatic ducts etc.) through which fluid, air or other substances (e.g., glandular secretions, milk, digestive or pancreatic enzymes, saliva etc.) are conveyed.

As used herein, the term “vortex,” “vortex flow” and/or “vortex fluid flow” refers to the laminar flow of fluids circumferentially along an interior surface of a cannula. Without wishing to be bound by any theory, a vortex is a fluid flow “enabler” established by differential fluid flow which generates curls in the velocity vector of the fluid (i.e., vorticities). By way of example, if one were to visualize a small portion of the fluid flowing through a cannula as a solid particle—while the remainder of the fluid flow disappears—the rotation of that solid particle would represent its vorticity. The differential flow resulting from these vortices generate higher circulation vectors at the center of the cannula and decreasing fluidic pressures at the walls of the cannula. The radial force of the resulting vortex “pushes” material inward away from the cannula wall, thereby enabling higher velocity fluid flow through the cannula. As disclosed herein, vortex flow through the cannula lumen may be “enhanced” by incorporating helically arranged dual friction surfaces within the inner wall of the cannula.

As used herein, the terms “spiral,” “helical” and grammatical equivalents thereof, may be used interchangeably to refer to a smooth curve along a three-dimensional space in which a tangent line at any point along the curve forms a constant angle with a fixed lit (i.e., the axis) of the curve. As used herein, the term “pitch” refers to the width of one complete turn of the spiral measured parallel to the axis of the spiral.

As outlined above, existing catheter systems may not be effective in removing undesirable materials from medium to large blood vessels or heart chambers due to their small size relative to the material to be removed. As a result, the undesirable material often needs to be fragmented to fit within the catheter lumen. Unfortunately, fragmentation increases the likelihood of potentially life-threatening downstream obstructions. Enlarging the catheter to accommodate larger undesirable materials may result in an unacceptable volume of blood being aspirated, resulting in excessive fluid loss and/or shock to the patient, or in a device that is too large to fit in the human anatomy.

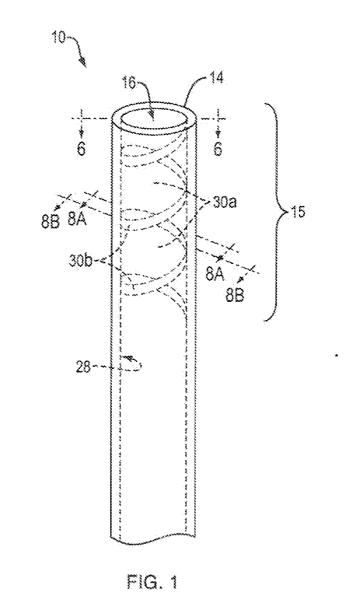

The systems and methods disclosed herein address the deficiencies of existing devices and techniques by allowing undesirable materials to be removed substantially en bloc from the vasculature, including medium to large blood vessels and chambers of the heart. Referring to FIG. 1, in one embodiment the present disclosure may include a system for removing undesirable material from within a vessel, comprising a suction cannula 10 that includes a proximal end not shown), a distal end 14 and a lumen 16 extending therebetween. The lumen 16 may be defined by an inner wall 28 that includes a substantially circular profile along a longitudinal axis of the suction cannula. The suction cannula 10 may be formed from a pliable material to allow the distal end 14 to be efficiently directed along a vessel to a site of interest within a patient. The suction cannula may also be reinforced (not shown) with wire or another suitable material to optimize maneuverability within the vessel without kinking. The suction cannula may be straight or may have a pre-bent configuration having an angle of up to 40 degrees in either direction relative to the central axis of the cannula. In addition, as the suction cannula may be used to apply a suction force to a site of interest to capture undesirable materials, the suction cannula may be formed from, or reinforced with, a sufficiently rigid material so as to prevent collapsing of the lumen under a suction force. In one embodiment, the suction cannula may be formed from a biocompatible material such as polyvinyl chloride, polyethylene, polypropylene, polyurethane, polyether block amide (i.e., Pebax®, silicone or combinations thereof.

To enhance vortex flow, the distal portion 15 of the suction cannula 10 may include dual-friction surfaces formed from first and second materials 30a, 30b arranged in a spiral (Le., helical) pattern within the inner wall 28, As discussed below, applying suction to the proximal end (not shown) of the suction cannula 10 urges fluid (i.e., blood) to flow through the distal end 14 and across the dual-friction surfaces 30a, 30b to generate an enhanced vortex flow that captures and removes undesirable materials en bloc, reducing the need to mechanically fracture the undesirable material prior to removal.

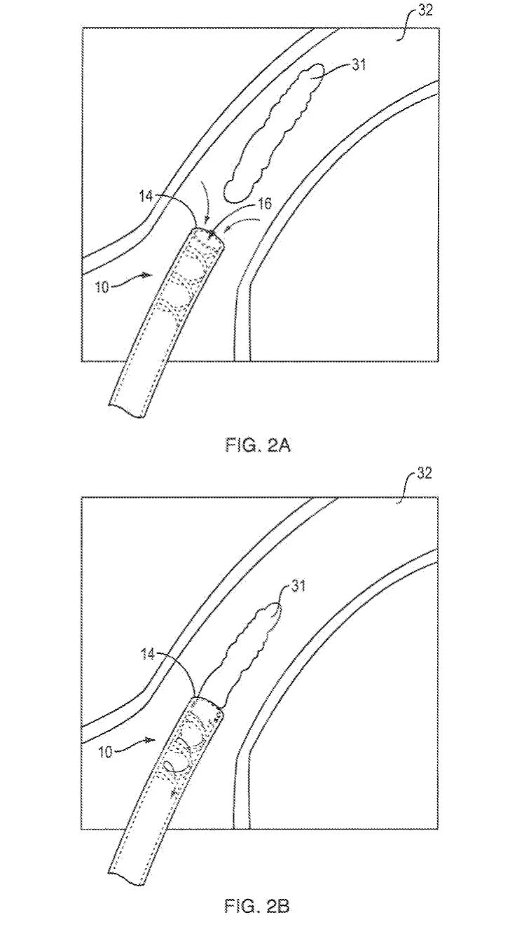

Referring to FIGS. 2A-2B, in use and by way of example, the suction cannula 10 may he advanced through the vasculature 32 of a patient such that the distal end 14 is positioned in contact with or adjacent to an undesirable material 31. Suction may then be applied to the proximal end (not shown) of the suction cannula 10 to generate a vortex flow of fluid (1, e., blood) through the distal portion of the cannula lumen 16 (FIG. 2A). As shown in FIG. 2B, the vortex flow directs the fluid and undesirable material 31 into the distal portion of the suction cannula 10 in a rotating/spiral path that carries the undesirable material 31 through the cannula lumen 16 to the proximal end (not shown). While the undesirable material 31 depicted in FIGS. 2A-2B is shown entering the cannula substantially en bloc, it should be appreciated that some undesirable materials, especially soft/fresh thrombi, may fragment in the presence of vortex flow to be removed in a series smaller portions. Thus, in addition to facilitating rotational flow of the undesirable material 31 through the cannula lumen 16, it should also be appreciated that the vortex flow ensures that the entirety of undesirable material 31 is directed into the cannula lumen 16 without the need of fragmentation.

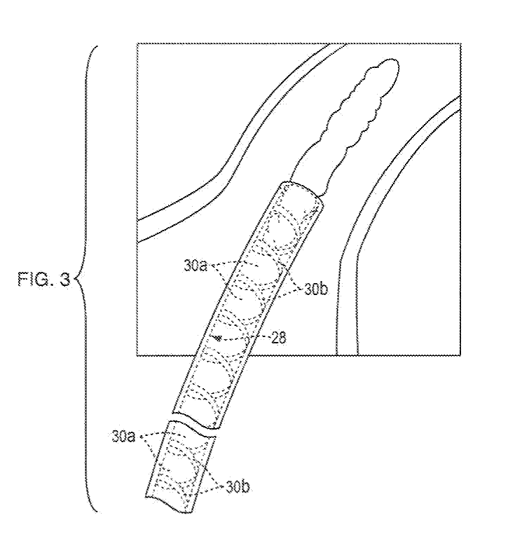

Referring to FIG. 3, in one embodiment the spiral pattern of first and second materials 30a, 30b within the inner wall 28 of the suction cannula may extend along the length of the cannula lumen rather than just the distal portion. Without wishing to be bound by any theory, it is believed that this additional spiraling maintains a sufficient flow rate throughout the length of the cannula lumen to ensure that the undesirable material does not become lodged therein prior to reaching the proximal end.

Although the undesirable material 31 depicted in FIGS. 2A-3 is approximately the same size (i.e., diameter) as the cannula lumen 16, it should be appreciated that in some instances the size of the undesirable material may be significantly larger than the cannula lumen. To better engage and capture the undesirable material substantially en bloc without significant fragmentation, in one embodiment the distal portion of the suction cannula 10 may be designed to include a variable-diameter opening 17 that expands according to the size of the undesirable material. For example, the distal portion of the suction cannula may be configured to assume a funnel-like shape that includes a maximum diameter opening of approximately at least three times that of the cannula lumen. Of course, depending on the size of the undesirable material being removed, the ratio between the diameter of the funnel and lumen may be varied.

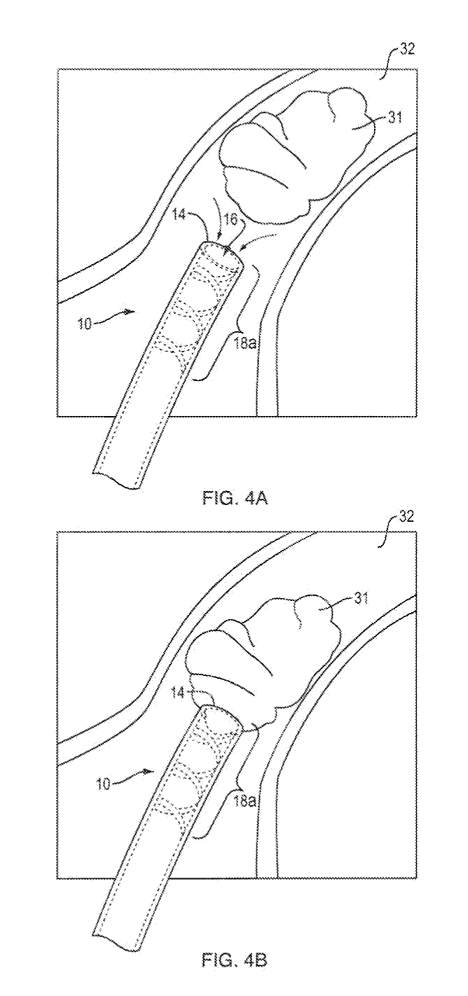

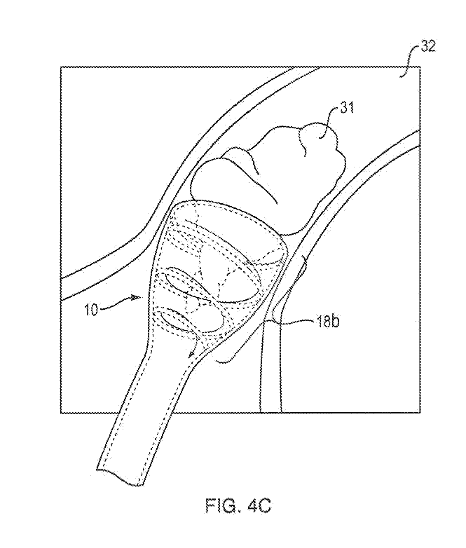

Referring to FIGS. 4A-4C, in one embodiment the distal portion of the suction cannula 10 may be advanced through the vasculature 32 of a patient in a first i.e., collapsed or unexpanded) configuration 18a and positioned in contact with or adjacent to an undesirable material 31 that exceeds the diameter of the cannula lumen 16. Suction may then be applied to the proximal end (not shown) of the suction cannula to generate vortex flow through the distal portion 15 (FIG. 4A). The vortex flow may pull a portion of the undesirable material 31 into the distal end 14 of the suction cannula resulting 10 in a temporary blockage of fluid flow through the cannula lumen 16 (FIG. 4B). This temporary blockage in fluid flow leads to a decrease in flow within the lumen 16 but a continued suction pressure within the cannula lumen 16 which urges the distal portion of the suction cannula to move from the first configuration 18a to a second (flared-open or expanded) configuration 18b (FIG. 4C), as discussed below. The flared-open configuration allows the distal portion of the cannula to accommodate the larger size of the undesirable material 31 and re-establish vortex flow to carry the undesirable material in a rotating/spiral path through the cannula lumen.

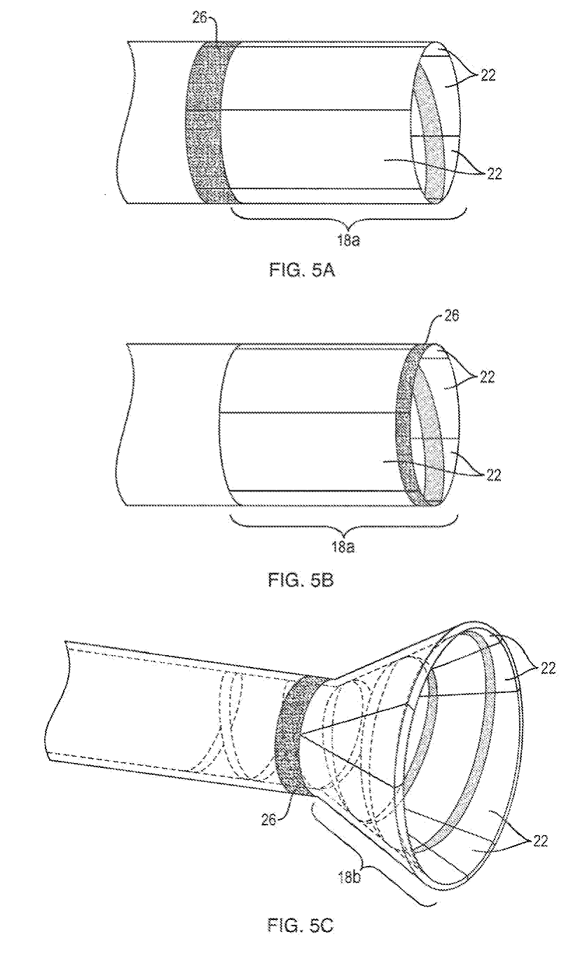

Referring to FIGS. 5A-5C, in one embodiment the variable-diameter opening at the distal portion of the suction cannula may include a plurality of independent curved strips 22, each coupled at one end to the distal end 14 of the suction cannula 10. Although FIGS. 5A-5C depict six curved strips coupled to the distal end of the cannula, it should be appreciated that that any number of curved strips may be used, if so desired. The curved strips 22 may be designed to pivot between the first configuration 18a (FIGS. 5A or 5B) in which the curved strips are substantially adjacent to one another, and the second configuration 18b (FIGS. 5C) in which the strips are flared-open in a funnel-like shape. As best illustrated in FIG. 5C, in one embodiment alternating strips 22 may be formed from a flexible material (i.e., webbing or impermeable membrane) to provide a consistent uninterrupted shape as the strips move from the first to second configuration. In another embodiment not shown), a continuous flexible/stretchable membrane may be circumferentially fixed (i.e., adhered, glued etc.) to the inner wall of each of the curved strips such that membrane stretches into a flared-open configuration as the strips move from the first to second configuration.

In one embodiment, the purpose of the first and second materials 30a, 30b of the present disclosure is to create a surface along the cannula inner wall 28 to promote or enhance the vortical flow, thereby promoting en bloc removal of undesirable material. Vortical flow may be enhanced by the first and second materials 30a, 30b in a number of ways, including, but not limited to, the following examples. The second material 30b may have a higher coefficient of friction than the first material 30a. Alternatively, the dual-friction surfaces may be formed from the same material, with the first material 30a having a substantially smooth surface and the second material 30b having a textured or rough surface e.g., stippling etc.). In another embodiment, the dual-friction surfaces may include one or more fluid channels (i.e., rifling or grooves) cut in a spiral pattern into the inner wall 28 at the distal portion of the suction cannula. In yet another embodiment, the fluid channel(s) may be formed from spirally arranged protrusions extending outward from the inner wall at the distal portion of the cannula. In certain embodiments, the inner wall of the cannula may include a single channel on the inner wall. In other embodiments the inner wall may include two or more channels. The number of channels may depend on a variety of factors, including, for example, the french size of the cannula and the width and height (or depth) of the channel(s). Alternative embodiments may include combinations of protrusions and channels to form the rifled inner wall at the distal portion 15 of the suction cannula. Rifled catheters and vascular access systems are known in the art, including, for example, U.S. patent application Ser. No. 14/447,002, herein incorporated by reference in its entirety.

The suction cannula of the present disclosure may be manufactured by a process in which first and second raw plastics or other suitable polymer materials are melted and co-extruded into the desired (i.e., tubular) shape. Alternatively, the suction cannula may be formed by a process in which a single plastic or other suitable polymer material is extruded into the desired (i.e., tubular) shape and channels formed within the inner wall using a die. The die is typically a metal structure with one or more openings shaped into the desired profile of the suction cannula. Molten polymer is pushed through the die and, as it exits the die, undergoes a cooling stage that causes the polymer to return to a solid form and retain the shape of the die opening. Spiraling protrusions or channels may be formed using a variety of methods known in the art, For example, as the molten polymer is pushed through the die, the die may rotate at a speed commensurate with the desired angle of the spiral. Alternatively, as the finished end of the cannula is pulled through the cooling stage, a pulling mechanism may rotate in a similar fashion to form the spiral effect.

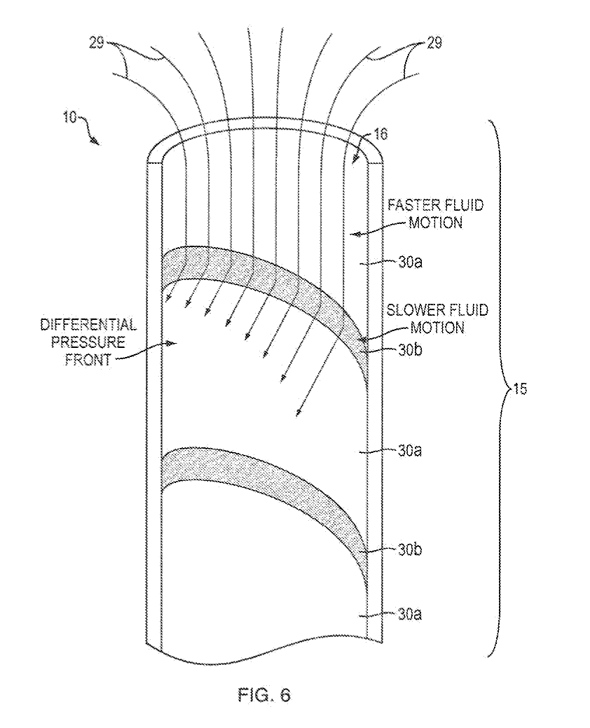

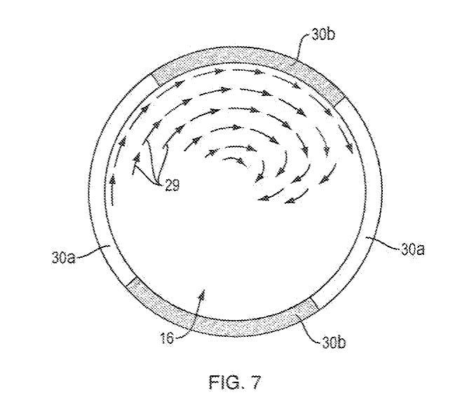

FIG. 6 depicts a cross-sectional view of the suction cannula 10 of FIG. 1 taken along the line 6-6. As fluid (i.e., arrows 29) enters the distal portion 15 of the suction cannula 10 under suction the fluid initially flows against the low coefficient of friction surface of the first material 30a. As the fluid continues through the cannula lumen 16 it flows against the higher coefficient of friction surface of the second material 30b and slows. As indicated by the direction of the arrows representing fluid flow 29, the spiral pattern of the first and second materials 30a, 30h causes the fluid flowing 29 through the distal portion 15 of the cannula lumen 16 to flow against the second (higher coefficient of friction) material 30b at different locations along, the longitudinal axis of the suction cannula 10. The fluid flowing 29 across the higher coefficient of friction surface of the second material 30b (i.e., the left side of the cannula) slows as compared to the corresponding portion of fluid that has not yet reached the second material (i.e., the right side of the cannula), thereby establishing a differential pressure front on one side (i.e., the left side) of the cannula lumen 16. Because this differential pressure front is created along the entire surface of the helically arranged first and second materials 30a, 30b, the fluid flowing 29 through the lumen 16 adjusts its path of flow to move laterally in the direction of the slower moving fluid (i.e., right to left). This lateral (i.e., rotational) flow increases as the fluid progresses through the cannula lumen 16 and flows against further windings of the second material 30b to generate a vortex flow that increases flow rate and funnels material toward the center of the cannula lumen 16 (FIG. 7).

In one embodiment, the spiral pattern may include a constant pitch (e.g., the pattern depicted in FIG. 1) in which the first and second materials 30a, 30b are evenly spaced along the axis of the cannula. In another embodiment, the spiral pattern may include a variable pitch (not shown) in which the spacing between the first and second materials 30a, 30b increases from the distal to proximal ends of the cannula. Stated differently, the spacing between the first and second materials 30a, 30b at the distal end of the cannula is less than (i.e., the spirals are closer together) the spacing between the first and second materials 30a, 30b at a proximal position along the axis of the cannula (i.e., the spirals are farther apart).

Still referring to FIG. 6, in one embodiment, the width of the higher coefficient of friction second material 30b relative to the corresponding lower coefficient of friction first material 30a along the distal portion 15 of the cannula may be 20 percent or less. It should be appreciated, however, that the dimensions of the first and second materials 30a, 30b may vary such that the width of the second material 30b relative to the first material is greater than 20 percent, Similarly in one embodiment, the width of the second material 30b may exceed that of the corresponding first material 30a.

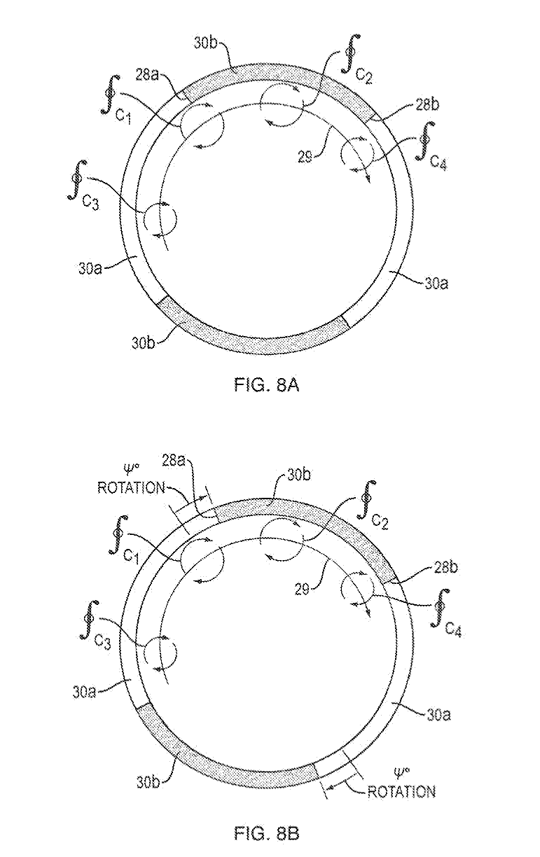

The rotational fluid flow through the cannula lumen may be further explained by reference to FIGS. 8A-8B which depict cross-sectional views of the suction cannula of FIG. 1 taken along differentially adjacent lines 8A-8A and 8B-8B, respectively. FIG. 8A-8B are intended to depict how the first and second materials 30a, 30b form a helical pattern along the longitudinal axis of the suction catheter. To help depict the helical pattern of the first and second materials 30a, 30b, FIG. 8A provides a relative starting point by which four exemplary closed loop fluid vectors (integrals §c₁-§c₄) are used to characterize fluid flow and vorticity at the low and high friction surfaces of the helically arranged first and second materials 30a, 30b. As discussed above, each of the four integrals represents the rotation of a portion of the fluid when perceived as a solid particle. The relative size of the circle defining each integral indicates the relative amount of rotation (i.e., the larger circles indicate more rotation and the smaller circles indicate less rotation). Referring to FIG. 8A, relative to the direction of fluid rotation (see arrow 29) the closed loop integral §c₁ is one-half within the radius angle of the leading edge 28a of the high friction surface of the second material 30b; closed loop integral §c₂ is located entirely Within the high friction surface of the second material 30b; closed loop integrals §c₃ is entirely within the low friction surface of the first material 30a; and closed loop integral §c₄ is entirely within the low friction surface of the first material 30a but on the nailing edge 28b of the high friction surface of the second material 301). FIG. 8B depicts the position of the high and low friction surfaces relative to integrals §c₁-§c₄ at a cross-section immediately downstream of the cross-section of FIG. 8A. As indicated by the fixed position of integrals §c₁-§c₄, the location of the high and low friction surfaces of the first and second material 30a 301) appears to have rotated by the angle ψ° such that the closed loop integral §c₁ is now located entirely within the low friction surface of the first material 30a, and the closed loop integral §c₄ is now located one-half within the trailing edge 28b of the high friction surface of the second material 30b. This depication of the integrals §c₁-§c₄ in FIGS. 8A-8B are for clarification purposes only and are intended to help visualize the helical path of the first and second materials 30a, 30b. it is understood that fluid would not remain stationary in such a way during actual use of Applicants device.

Because the value of each closed loop integral is unaltered as they “move” from surface to surface through a fluid, the vortex lines are shifted as each cross-section is evaluated from layer to layer. This means that each successive cross-sectional layer rotates the vortex column in step with the rotation or pitch (ψ°) of that layer. Applying this closed integral analysis to every location in a cross-sectional plane provides a change in rotational fluid velocity in each plane. When taken together, the differential fluid flow resulting from the vortices (i.e., integrals generated alone the lines 8A-8A (FIG. 8A) and 8B-8B (FIG. 8B) decreases fluidic pressure at the cannula walls and increases fluid circulation toward the center of the cannula. The radial force of the resulting vortex “pushes” material inward away from the cannula wail, thereby enabling higher velocity fluid flow through the cannula (FIG. 7).

It should be appreciated that the suction cannula described herein may be scaled from small to large sizes (i.e., 8-22 french) while retaining sufficient fluid velocity and vortex flow to remove undesirable materials substantially en bloc from a variety of different sized vessels without significant fragmentation. In general, to avoid shearing cellular components of the blood, a cannula having an outer diameter of 22 french should support a fluid velocity capable of evacuating approximately 6.0 liters of blood per minute. By comparison, a cannula having an outer diameter of 8 french should support a fluid velocity capable of evacuating approximately 0.48 liters per minute to avoid shearing cellular components of the blood.

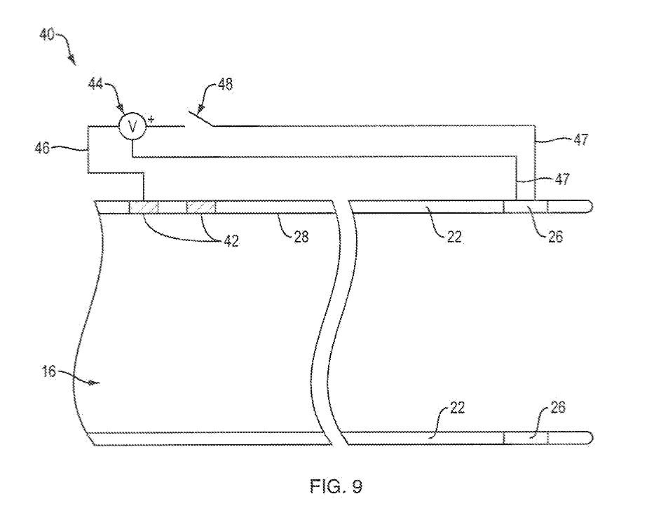

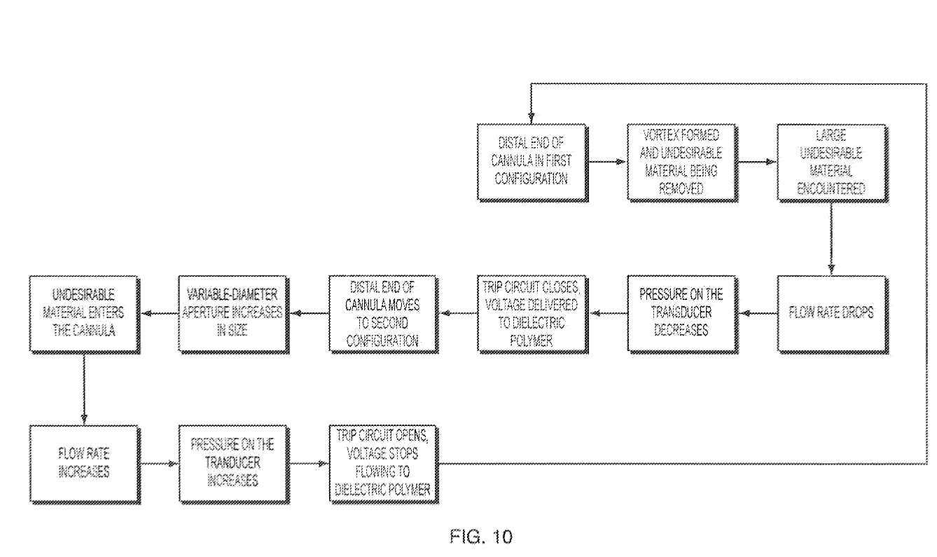

Referring again to FIGS. 5A and 5B, to deploy the strips 22 between the first and second configurations, a portion of the strips may include a dielectric polymer 26 that changes in shape when stimulated by an electric field to move between the first and second configurations 18a, 18b without the need for user interaction or computerized assistance. As illustrated in FIG. 9, movement of the strips 22 between the first and second configurations may be controlled by an automated electromechanical feedback loop 40. The electromechanical feedback loop may include a downstream pressure transducer senor 42 located within a portion of the cannula inner wall 28 that directly or indirectly monitors fluid flow through the cannula lumen 16. A voltage source 44 may be electrically connected to the pressure transducer sensor 42 by a first wire 46 and to the dielectric polymer 26 by a second set of wires 47 which form a circuit that includes a switch or trip circuit 48. With reference to the flow chart of FIG. 10, the distal end of the cannula may be positioned adjacent to an undesirable material with a diameter than exceeds the diameter of the cannula lumen. Suction may be applied to the proximal end of the cannula to establish vortex flow such that the fluid (i.e., blood) and undesirable material flow through the cannula lumen in a vortical direction. As the distal end of the cannula becomes obstructed by a portion of the undesirable material that is larger than the diameter of the cannula lumen, the fluid flow rate through the cannula lumen decreases below a preset threshold velocity. The threshold velocity can be set by the user and can be any desired flow rate in which the user desires. The accompanying decrease in fluid pressure is detected by the pressure transducer sensor 42 which sends an electrical signal along the first wire 46 to the voltage source 44. In response to this electrical signal the voltage source 44 closes the trip circuit 48 to deliver electrical energy from the voltage source 44 to dielectric polymers 26 through the second set of wires 47. The voltage passing through the dielectric polymers 26 moves the strips 22 from the first (unexpanded) configuration to a second (flared-open) configuration. The strips continue to move to a flared-open configuration until the variable-diameter opening is sufficiently large to pull the undesirable material into the cannula lumen and restore vortex flow. As the fluid and undesirable material move through the cannula lumen (not shown) the fluid flow rate and accompanying fluid pressure within the cannula lumen returns to normal (i.e., above the threshold) level. The pressure transducer sensor 42 detects this pressure increase and stops sending the electrical signal to the voltage 44 source. The voltage source 44 then opens the trip circuit 48 to stop the flow of electrical current to the dielectric polymers 26 such that the strips 22 return to the first configuration.

It should be appreciated that the present disclosure is not limited to the use of dielectric polymers to control the movement of the strips 22 between the first and second configurations. For example, in one embodiment the strips 22 (or a portion thereof) may be formed from one or more shape-memory metals or metal alloys that move from a first to second (i.e., memory shape) in response to heat (e.g., an electric field which creates a dual heating effect). Non-limiting examples of such metals or metal alloys may include platinum group metals, particularly platinum, rhodium, palladium, and rhenium, as well as tungsten, gold, silver, tantalum, and alloys of these metals including platinum alloys (e.g., a platinum-tungsten alloy), as well as stainless steel, nickel-titanium alloys (nitinol), and Elgiloy® (from Elgiloy Specialty Metals).

Although the electromechanical feedback loop acts without intervening software to actuate the dielectric polymer, in one embodiment the electromechanical control loop may include a software platform which monitors the pressure transducer and moves the strips between the open and closed configurations independent of the voltage circuitry.

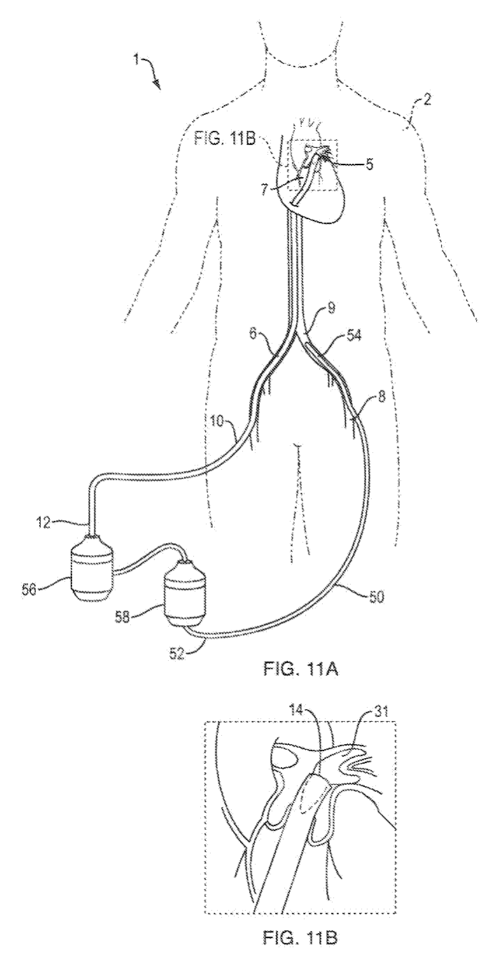

In one embodiment the suction cannula described herein may he a component of a system 1 for removing undesirable materials substantially en bloc from an obstruction site within the vasculature and reinfusing the removed e., suctioned or aspirated) fluid back into the patient to minimize fluid loss. Such aspiration and reinfusion systems are known in the art, including, for example, U.S. Pat. No. 8,075,510, herein incorporated by reference in its entirety. Referring to FIGS. 11A-11B, the system 1 may include the suction cannula 10, a reinfusion cannula 50, a pump 56 and a filter device 58. The distal portion 15 of the suction cannula 10 may be advanced through the vasculature 32 of a patient 2 to a site of interest 5, such as a chamber of the heart (FIG. 11B) that includes an undesirable material 31. The proximal end 12 of the suction cannula 10 may be fluidly connected to a filter device 58. The filter device may include an inlet (not shown) through which fluid removed from the site of interest along with the captured undesirable material can be directed from the suction cannula. The filter device may also include an outlet (not shown) through which filtered fluid may be directed to prevent the undesirable material captured from the site of interest from moving downstream of the system.

The filter device 10 may be fluidly connected to a pump 56 configured to generate the negative pressure (i.e., suction) required to establish the vortex flow which pulls the undesirable material from the site of interest 5 into the suction cannula 10. In one embodiment, the pump may include an intake port (not shown) in fluid communication with the outlet of the filter device. The pump 56 may also be configured to generate the positive pressure required to provide the driving force which directs the aspirated fluid through an exit port (not shown) and through the reinfusion cannula 50 to return the fluid removed from the site of interest back into the patient. In one embodiment, the suction force and the driving force may be generated by the pump simultaneously and may take place continuously or intermittently for a set duration. The pump may include any commercially available pump, including those used for medical applications and those capable of pumping fluids, such as blood. Examples of such a pump may include a kinetic pump, such as a centrifugal pump, and an active displacement pump, such as a rollerhead pump.

The reinfusion cannula 50 may include a proximal end 52, a distal end 54 and a lumen (not shown) extending therebetween. The distal end 54 of the reinfusion cannula 50 may be advanced through the vasculature of the patient 2 to a desired site 5 and the proximal end 52 of the reinfusion cannula 50 may be fluidly connected to the exit port of the pump 56. Cleansed or filtered fluid may be directed from the filter device by way of the pump through the lumen of the reinfusion cannula to be reinfused into the patient at the desired site. To that end, the reinfusion cannula may be configured for positioning within the same or different vessel within which the suction cannula may be positioned. in one embodiment, the distal end of the reinfusion cannula may be configured so that it can be positioned in spaced relation to the distal end of the suction cannula when the system is in operation.

The reinfusion cannula may be configured to be introduced into, and maneuvered through, the vasculature. Similar to the suction cannula, the reinfusion cannula may be formed from a pliable material. For example, the reinfusion cannula may be formed from a biocompatible material, such as polyvinyl chloride, polyethylene, polypropylene, polyurethane, Pebax®, silicone or combinations thereof Since reinfusion cannula may be made from a pliable material, to efficiently direct it along a vessel to the reinfusion site, the reinfusion cannula may be reinforced to optimize maneuverability within the vessel without kinking.

Still referring to FIGS. 11A-11B, in general the method of the present disclosure may include initially accessing a first vessel 6 either by surgical dissection or percutaneously with, for instance, a needle and guide wire. The blood vessel through which suction cannula 10 may be inserted into patient 2 may be any blood vessel capable of being accessed percutaneously or by surgical dissection such as femoral vein, femoral artery or jugular vein. The suction cannula 10 may be inserted into the first blood vessel 6 over the guide wire, and advanced toward a site of interest 5, for instance, in a second vessel or a heart chamber 7 where an undesirable material 31 may be residing. The second blood vessel or heart chamber may include the main pulmonary artery, branch pulmonary arteries, inferior versa cavae, superior vena cavae, deep veins of the pelvic, legs, arms or neck, aorta, or any other medium to large blood vessel for which the use of a cannula is suitable for removing undesirable material without causing undesirable damage to the blood vessel. In addition, the advancement of suction cannula 10 may be gauged or documented by fluoroscopic angiography, echocardiography or other suitable imaging modality.

In the case of pulmonary embolism, the suction cannula 10 may normally be introduced through the femoral, jugular or subclavian vein. Alternatively, the suction cannula 10 may be introduced directly into the cardiac chambers using a minimally invasive surgical or endoscopic, thoracoscopic, or pericardioscopic approach.

Thereafter, a third vessel 8 may be accessed either by surgical dissection or percutaneously with, for example, a needle and guide wire. The reinfusion cannula 50 may than be inserted into the third vessel 8 using an open or over the guide wire technique. The third blood vessel through which the reinfusion cannula 50 may be inserted may include any large vein, such as the femoral vein or jugular vein. The reinfusion cannula 50 may then be advanced toward a reinfusion she, for example, a fourth vessel 9 such as the femoral vein, iliac vein, inferior vena cava, superior vena cava or right atrium.

Once the reinfusion cannula 50 is in place and components of system 1 have been connected, the pump 56 may be activated, and suction cannula 50 placed against and in substantial engagement with the undesirable material 31 at the site of interest 5 for removal by suctioning through the suction cannula 10. The undesirable material 31 and circulatory fluid removed from the site of interest 5 may thereafter be directed along suction cannula 10 into filter device 58 where the undesirable material 31 can be entrapped and removed from the fluid flow. The resulting, filtered fluid may next be directed downstream by way of the pump 56 into the second filter device (not shown), where additional debris or material (e.g., ranging from smaller than microscopic in size to relatively larger) that may have escaped and moved downstream from filter device 58 can be further captured and removed from the fluid flow prior to reinfusion. The resulting cleansed fluid may then be directed into the reinfusion cannula 50 and introduced back into the patient 2.

All of the systems, assemblies and/or methods disclosed and claimed herein can be made and executed without undue experimentation in light of the present disclosure. While the present invention have been described in terms of preferred embodiments, it will be apparent to those of skill in the art that variations can be applied to the systems, assemblies and/or methods described herein without departing from the concept, spirit and scope of the invention. All such similar substitutes and modifications apparent to those skilled in the art are deemed to be within the spirit scope and concept of the invention as defined by the appended claims.

While embodiments of the invention have been illustrated and described, it is not intended that these embodiments illustrate and describe all possible forms of the invention Rather, the words used in the specification are words of description rather than limitation, and it is understood that various changes may be made without departing from the spirit and scope of the invention.

What is claimed is:

1. A system for removing an undesirable material from within a vessel, the apparatus comprising:
a cannula having a proximal end, a distal end and a lumen extending therebetween;
wherein a distal portion of the cannula is moveable between a first configuration and a second configuration larger than the first configuration; and
wherein the distal portion of the cannula includes an inner wall comprising a first material having a first coefficient of friction and a second material having a second coefficient of friction different than the first material.

2. The system of claim 1, wherein the the first and second materials are arranged in a spiral, and the distal portion of the cannula is configured to generate vortex flow therethrough.

3. The system of claim 2, wherein the undesired material includes one of a clot, a thrombus and an embolus, and wherein the vortex flow facilitates en bloc removal of the undesirable material through the cannula.

4. The system of claim 1, wherein the second configuration of the distal portion of the cannula is configured to engage and capture the undesirable material.

5. The system of claim 1, wherein the second configuration of the distal portion of the cannula includes a funnel-like shape.

6. The system of claim 1, wherein the distal portion of the cannula moves from the first configuration to the second configuration when a fluid flow rate through the cannula lumen decreases below a threshold value.

7. The system of claim 1, wherein the distal portion of the cannula moves from the first configuration to the second configuration when a pressure within the cannula lumen decreases below a threshold value.

8. The system of claim 6, wherein the distal portion of the cannula returns to the first configuration when a fluid flow rate through the cannula lumen returns to a threshold value.

9. The system of claim 7, wherein the distal portion of the cannula returns to the first configuration when a pressure within the cannula lumen returns to a threshold value.

10. The system of claim 1, wherein the distal portion of the cannula includes a dielectric polymer, and wherein the distal portion of the cannula moves from the first configuration to the second configuration in response to an increase in voltage.

11. The system of claim 1, wherein at least a portion of an inner wall of the cannula includes a surface comprising a first material having a first coefficient of friction and a second material having a second coefficient of friction different than the first material, wherein the first and second materials are arranged in a spiral pattern.

12. The system of claim 1, wherein the cannula is formed from a sufficiently pliable material to permit maneuvering within the vessel.

13. The system of claim 1, wherein the second material further consists of a grove along the inner wall.

14. A system for removing an undesirable material from within a vessel, the system comprising:
a first cannula having a proximal end, a distal end and a Lumen extending therebetween;
wherein a distal portion of the cannula is moveable between a first configuration and a second configuration larger than the first configuration; and
wherein the distal portion of the cannula includes an inner wall comprising a first material having a first coefficient of friction and a second material having a second coefficient of friction different than the first material, wherein the first and second materials are arranged in a spiral pattern;
a second cannula in fluid communication with the first cannula and having its distal end situated in spaced relation to the distal end of the first cannula, such that fluid removed from the site of interest by the first cannula can be directed along the second cannula and reinfused through the distal end of the second cannula; and
a pump, in fluid communication with the proximal end of the first cannula to provide a sufficient suction force for generating a vortex flow through the expanded distal and to permit removing the undesirable material from the site of interest, while in fluid communication with the second cannula, to provide a sufficient continuous reinfusion force so as to reintroduce the fluid flow removed from the site of interest.

15. A method for removing an undesirable material from within as vessel, the method comprising:
maneuvering a first cannula having a distal end through which an undesirable material can be captured to a site of interest within a vessel of a patient, such that the distal end of the first cannula is positioned adjacent the undesirable material;
positioning a second cannula, in fluid communication with the first cannula, such that its distal end is situated in spaced relation to the distal end of the cannula;
providing a suction force to generate a vortex flow through the distal end of the first cannula, so as to remove the undesirable material en bloc from the first cannula; and
while providing the suction force to the first cannula, continuously imparting a reinfusion force through the distal end of the second cannula, so that any fluid remove along with the undesirable material by the first cannula can be reintroduced by the second cannula to a location in spaced relation form the distal end of the first cannula.

15. The method of claim 15, wherein the distal end of the first cannula expands from a first diameter to a second diameter larger than the first diameter to remove large embolic emboli.
